(19) United States
(12) Patent Application Publication
(10) Pub. No.: US 2021/0265027 A1
(43) Pub. Date: Aug. 26, 2021

(54) METHOD AND SYSTEM FOR CONDUCTING SURVEY RELATING TO URINATION, AND NON-TRANSITORY COMPUTER-READABLE RECORDING MEDIUM

(71) Applicant: DAIN TECHNOLOGY, INC., Seoul (KR)

(72) Inventors: Kyeong Yeon DOO, Seoul (KR); Jee Young SONG, Seoul (KR); Ji Young JUNG, Yongin-si, Gyeonggi-do (KR)

(73) Assignee: DAIN TECHNOLOGY, INC., Seoul (KR)

(21) Appl. No.: 17/256,351

(22) PCT Filed: Jun. 28, 2019
(86) PCT No.: PCT/KR2019/007902

§ 371 Date: Mar. 2, 2021

(30) Foreign Application Priority Data
Jun. 29, 2018 (KR) 10-2018-0075209

(51) Int. Cl. G16H 10/20; G06F 3/16

(52) U.S. Cl. CPC G16H 10/20; G06F 3/16

(57) ABSTRACT

According to one aspect of the present invention, there is provided a method for conducting a survey on urination, the method comprising the steps of: determining at least one survey item to be provided to a user when an event related to the user's urination occurs, with reference to context information on the event; and acquiring the user's answer corresponding to the at least one survey item.

### CROSS-REFERENCE TO RELATED APPLICATION(S)

This is a national phase of Patent Cooperation Treaty (PCT) International Application No. PCT/KR2019/007902 filed on Jun. 28, 2019, which claims priority to Korean Patent Application No. 10-2018-0075209 filed on Jun. 29, 2018. The entire contents of PCT International Application No. PCT/KR2019/007902 and Korean Patent Application No. 10-2018-0075209 are hereby incorporated by reference.

### FIELD OF THE INVENTION

The present invention relates to a method, system, and non-transitory computer-readable recording medium for conducting a survey on urination.

This application is based on the project conducted as follows:

Project Identification Number: 9991006814, KMDF_PR_20200901_0141

Ministry Name: Ministry of Science and ICT, Ministry of Trade, Industry and Energy, Ministry of Health and Welfare, and Ministry of Food and Drug Safety of Korea

Research Program Title: Cross-Ministry Full Cycle Medical Device Research and Development Program

Research Project Title: Development of Artificial Intelligence-based Digital Treatment for Micturition Disorders

Research Management Institution: Cross-Ministry Full Cycle Medical Device Research and Development Program Foundation

Contribution Rate: 50%

Host Research Institute: Bundang Hospital of Seoul National University

Research Period: Sep. 1, 2020 through Dec. 31, 2023

### BACKGROUND

In urology departments, urination survey tests (e.g., the international prostate symptom score (IPSS) test, the overactive bladder symptom score (OABSS) test, etc.) are commonly conducted for patients with symptoms such as benign prostatic hyperplasia and overactive bladder, as well as patients with frequent urination, nocturia, or urinary incontinence. The urination survey tests provide important basic data for allowing a doctor to objectively identify a patient's urination symptoms and conditions and make an accurate diagnosis.

However, for the urination survey tests according to the techniques introduced so far as well as the above-described conventional technique, patients should visit a hospital and record all their urinary symptoms of the last few days relying only on their memory, which makes it virtually difficult to acquire accurate results of the tests. Further, the urination survey tests include not only survey items to be answered at a predetermined period but also survey items to be answered at each time of urination. In addition, there may be survey items to be answered only in a specific urination situation, among the survey items to be answered at each time of urination. Thus, even if the patients take the urination survey tests without visiting the hospital, it may be uncomfortable and cumbersome to check the predetermined period or the specific urination situation at each time of urination.

In this connection, the inventor(s) present a technique capable of analyzing context information on an event related to a user's urination when the event occurs to determine survey items that require the user's answers, and collecting the user's answers corresponding to the determined survey items.

### SUMMARY OF THE INVENTION

One object of the present invention is to solve all the above-described problems in the prior art.

Another object of the invention is to efficiently conduct a urination survey test by providing a user with survey items suitable for a urination situation and acquiring answers thereto.

Yet another object of the invention is to allow a survey on urination to be conveniently conducted by estimating answers corresponding to some survey items on the basis of context information on an event related to a user's urination.

Still another object of the invention is to improve the accuracy and reliability of a result of a urination survey test by allowing survey items to be provided when (or immediately after) a urination event occurs.

The representative configurations of the invention to achieve the above objects are described below.

According to one aspect of the invention, there is provided a method for conducting a survey on urination, the method comprising the steps of: determining at least one survey item to be provided to a user when an event related to the user's urination occurs, with reference to context information on the event; and acquiring the user's answer corresponding to the at least one survey item.

According to another aspect of the invention, there is provided a system for conducting a survey on urination, the system comprising: a survey item determination unit configured to determine at least one survey item to be provided to a user when an event related to the user's urination occurs, with reference to context information on the event; and an answer acquisition unit configured to acquire the user's answer corresponding to the at least one survey item.

In addition, there are further provided other methods and systems to implement the invention, as well as non-transitory computer-readable recording media having stored thereon computer programs for executing the methods.

According to the invention, it is possible to efficiently conduct a urination survey test by providing a user with survey items suitable for a urination situation and acquiring answers thereto.

According to the invention, it is possible to allow a survey on urination to be conveniently conducted by estimating answers corresponding to some survey items on the basis of context information on an event related to a user's urination.

According to the invention, it is possible to improve the accuracy and reliability of a result of a urination survey test by allowing survey items to be provided when (or immediately after) a urination event occurs.

### BRIEF DESCRIPTION OF THE DRAWINGS

FIG. 1 specifically shows the internal configuration of a urination survey system according to one embodiment of the invention.

FIG. 2 illustratively shows how to conduct a survey on urination according to one embodiment of the invention.

FIG. 3 illustratively shows a survey on urination according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures, and characteristics described herein may be implemented as modified from one embodiment to another without departing from the spirit and scope of the invention. Furthermore, it shall be understood that the positions or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the present invention will be described in detail with reference to the accompanying drawings to enable those skilled in the art to easily implement the invention.

Configuration of a Urination Survey System

Hereinafter, the internal configuration of a urination survey system 100 crucial for implementing the invention and the functions of the respective components thereof will be discussed.

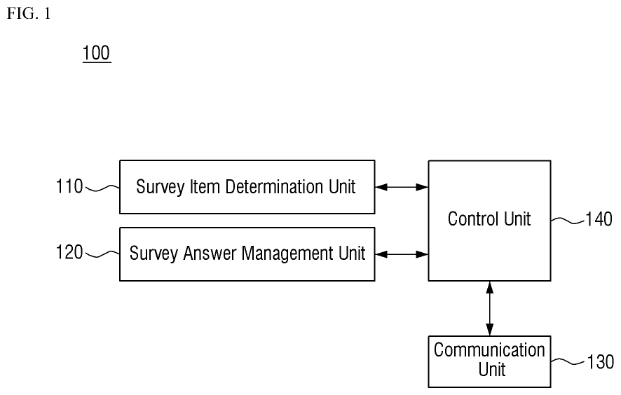

FIG. 1 specifically shows the internal configuration of the urination survey system 100 according to one embodiment of the invention.

Referring to FIG. 1, the urination survey system 100 according to one embodiment of the invention may comprise a survey item determination unit 110, a survey answer management unit 120, a communication unit 130, and a control unit 140. According to one embodiment of the invention, at least some of the survey item determination unit 110, the survey answer management unit 120, the communication unit 130, and the control unit 140 may be program modules that communicate with an external system (not shown). The program modules may be included in the urination survey system 100 in the form of operating systems, application program modules, and other program modules, while they may be physically stored in a variety of commonly known storage devices. Further, the program modules may also be stored in a remote storage device that may communicate with the urination survey system 100. Meanwhile, such program modules may include, but are not limited to, routines, subroutines, programs, objects, components, and data structures for performing specific tasks or executing specific abstract data types according to the invention as will be described below.

Meanwhile, the above description is illustrative although the urination survey system 100 has been described as above, and it will be apparent to those skilled in the art that at least a part of the functions or components of the urination survey system 100 may be implemented or included in a device 200 to be described, as necessary. Further, in some cases, all the functions and components of the urination survey system 100 may be implemented or included in the device 200.

First, the survey item determination unit 110 according to one embodiment of the invention may determine at least one survey item to be provided to a user when an event related to the user's urination occurs, with reference to context information on the event. According to one embodiment of the invention, the event related to the urination may be detected by a sound signal related to the urination, a video signal related to the urination, a signal inputted by the user (e.g., a voice trigger, a button trigger, or a gesture trigger), and the like, and may include an event related to a start, end, stop, urine flow rate, voided urine volume, or the like of the urination. Further, according to one embodiment of the invention, the context information on the event may include information on a start or end point, circumstances, or a pattern (e.g., a pattern in which the urination is repeated at a specific time, or the voided urine volume is greater at night than in the morning) of the event related to the urination.

Specifically, the survey item determination unit 110 according to one embodiment of the invention may determine at least one survey item among a plurality of survey items related to the user's urinary health, with reference to context information on the event related to the user's urination. According to one embodiment of the invention, the plurality of survey items related to the user's urinary health may be determined on the basis of information on the user's urinary health. In this case, the information on the user's urinary health according to one embodiment of the invention may be acquired by analyzing the event related to the user's urination, acquired from information on a doctor's diagnosis for the user, or inputted by the user. In addition, the survey item determination unit 110 according to one embodiment of the invention may determine the plurality of survey items related to the user's urinary health with reference to survey items on various urinary symptoms, such as survey items of the international prostate symptom score (IPSS), the overactive bladder symptom score (OABSS), and the patient perception of bladder condition (PPBC).

For example, the survey item determination unit 110 according to one embodiment of the invention may determine the IPSS survey items as the plurality of survey items related to the user's urinary health. Further, when there occurs an event in which the user's urination is started, the survey item determination unit 110 according to one embodiment of the invention may determine, on the basis of context information on the urination (which is determined from a start time of the event, an end time of the event, whether the urination is stopped during the event, and the like), survey items related to the context information, among the IPSS survey items (i.e., the plurality of survey items), as the survey items to be provided to the user. In this case, according to one embodiment of the invention, when the event in which the user's urination is started occurs in the user's bedtime hours, the context information may indicate that the urination is nocturia, and when there occurs an event in which the user's urination is temporarily stopped for a predetermined period of time after the urination is started, the context information may indicate that the user tightens his/her stomach because the urine does not come out well.

As another example, the survey item determination unit 110 according to one embodiment of the invention may recognize the event related to the user's urination on the basis of a sound signal related to the urination. Specifically, the survey item determination unit 110 according to one embodiment of the invention may estimate numerical urination information of the user, such as a urine flow rate (or a voided urine volume per hour), a maximum urine flow rate, a start point of the urination, an end point of the urination, and a voided urine volume, on the basis of a sound signal related to the user's urination (specifically, on the basis of urination parameters related to an effective (or a root mean square; RMS) value and a spectral centroid calculated from the sound signal), and may determine the at least one survey item to be provided to the user with reference to context information on the event related to the urination, which is determined on the basis of the numerical urination information. As a more detailed example, the survey item determination unit 110 according to one embodiment of the invention may recognize an event related to a start of the user's urination (i.e., whether the urination is started) on the basis of a sound signal related to the user's urination, and when there occurs an event in which the user's maximum urine flow rate estimated on the basis of the sound signal is 15 ml/sec or less, the survey item determination unit 110 may determine the at least one survey item to be provided to the user with reference to context information on the event, which may indicate that the urinary stream is weak.

Meanwhile, the survey item determination unit 110 according to one embodiment of the invention may specify context information on the event related to the urination by analyzing information on at least one of the user's schedule, text chats, messages, social networking service (SNS) chats, e-mail, and applications (e.g., healthcare applications) acquired from the device 200 of the user, and may determine the at least one survey item provided to the user with reference to the specified context information.

For example, when an event in which the user's urination is started occurs at night (e.g., 11 p.m.), the survey item determination unit 110 according to one embodiment of the invention may specify context information indicating that the urination is nocturia, on the basis of the user's bedtime (e.g., 10 p.m.) acquired from the device 200 of the user, and may determine the at least one survey item provided to the user with reference to the specified context information.

Further, the survey item determination unit 110 according to one embodiment of the invention may estimate an answer corresponding to at least one survey item other than the at least one determined survey item, among the plurality of survey items related to the user's urinary health, with reference to the context information on the event.

For example, the survey item determination unit 110 according to one embodiment of the invention may estimate the user's answers to the remaining survey items other than the at least one determined survey item, among the plurality of survey items related to the user's urinary health, by analyzing the context information on the event related to the user's urination without directly acquiring the user's answers to the remaining survey items.

Next, the survey answer management unit 120 according to one embodiment of the invention may acquire the user's answer corresponding to the at least one survey item determined by the survey item determination unit 110.

Specifically, the survey answer management unit 120 according to one embodiment of the invention may provide the user with the at least one determined survey item and acquire the user's answer corresponding to each of the at least one provided survey item.

For example, the survey answer management unit 120 according to one embodiment of the invention may provide the user with the at least one determined survey item when (or immediately after) the event related to the user's urination is ended. That is, according to one embodiment of the invention, the accuracy of a result of the survey on urination may be improved by providing appropriate survey items suitable for the urination situation as soon as the event related the user's urination is ended.

Meanwhile, the survey answer management unit 120 according to one embodiment of the invention may provide the user with an estimated answer to at least one of the survey items to be provided to the user, with reference to the context information on the event.

For example, when a survey item to be provided to the user is “How often have you had a weak urinary stream?” and the context information on the event related to the user's urination indicates that the weak urinary stream occurred three times in a week, the survey answer management unit 120 according to one embodiment of the invention may provide “About half the time” as an estimated answer, among the answer options to be provided to the user in correspondence to the survey item, i.e., “Almost always”, “About half the time”, “More than half the time”, and “Not at all”.

Further, the survey answer management unit 120 according to one embodiment of the invention may produce an answer analysis result of the survey on the user's urination with reference to the user's answer corresponding to the at least one survey item.

For example, the survey answer management unit 120 according to one embodiment of the invention may calculate a survey score corresponding to each of the IPSS or OABSS survey items at a predetermined period or at each time of urination, with reference to the user's answer corresponding to the at least one survey item, and may produce at least one of an average, a maximum value, and a minimum value of the scores as the answer analysis result.

Further, the survey answer management unit 120 according to one embodiment of the invention may determine the user's urinary health status with reference to the produced answer analysis result of the survey on the user's urination.

For example, the survey answer management unit 120 according to one embodiment of the invention may acquire an average survey score of the user corresponding to the IPSS survey items from the answer analysis result of the survey on the user's urination, and may determine that the user's urinary health status is abnormal when the average survey score is not greater than a predetermined level for a predetermined period of time.

Next, the communication unit 130 according to one embodiment of the invention may function to enable data transmission/reception from/to the survey item determination unit 110 and the survey answer management unit 120.

Lastly, the control unit 140 according to one embodiment of the invention may function to control data flow among the survey item determination unit 110, the survey answer management unit 120, and the communication unit 130. That is, the control unit 140 according to the invention may control data flow into/out of the urination survey system 100 or data flow among the respective components of the urination survey system 100, such that the survey item determination unit 110, the survey answer management unit 120, and the communication unit 130 may carry out their particular functions, respectively.

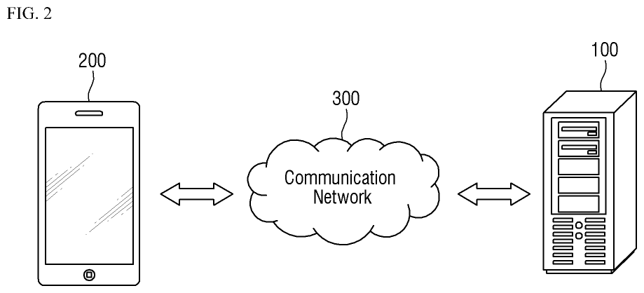

FIG. 2 illustratively shows how to conduct a survey on urination according to one embodiment of the invention.

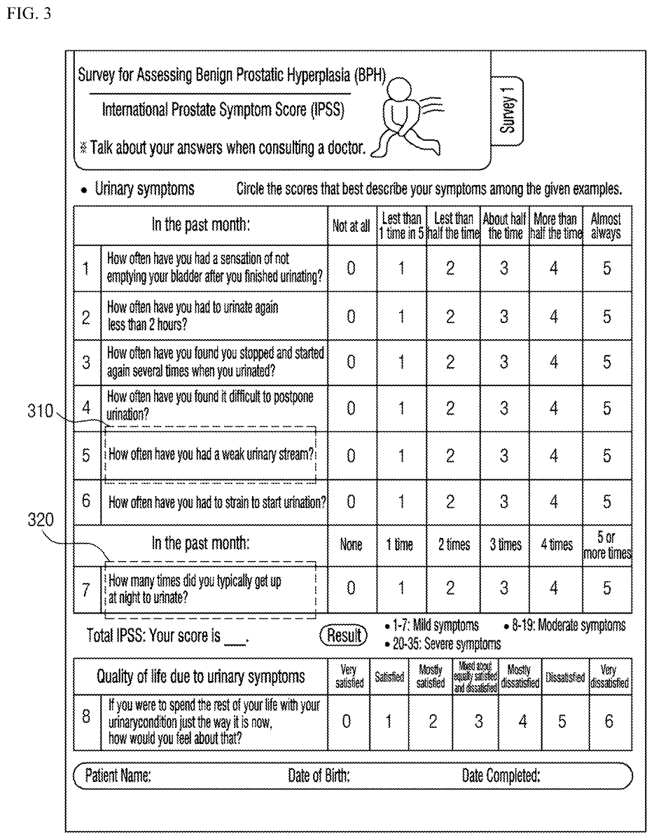

FIG. 3 illustratively shows a survey on urination according to one embodiment of the invention.

Referring to FIG. 2, it may be assumed that a server system 100 including the urination survey system 100 according to the invention is interconnected with a device 200 of a user via a communication network 300.

The device 200 according to one embodiment of the invention is digital equipment having a memory means and a microprocessor for computing capabilities, and may include a wearable device such as smart glasses, a smart watch, a smart band, a smart ring, a smart necklace, a smart earset, smart earphones, and smart earrings, or include a somewhat traditional device such as a smart phone, a smart pad, a desktop computer, a notebook computer, a workstation, a personal digital assistant (PDA), a web pad, a mobile phone, and a remote controller. Besides the foregoing examples, the device 200 may be changed without limitation as long as the objects of the invention may be achieved. Further, the device 200 according to one embodiment of the invention may include a sound acquisition (or sound detection) module (e.g., a microphone) for acquiring a sound signal (e.g., a sound signal related to urination).

Meanwhile, the device 200 according to one embodiment of the invention may include an application for supporting the functions of conducting a survey on urination according to the invention. The application may be downloaded from the urination survey system 100 according to the invention or an external application distribution server (not shown). The characteristics of the program module may be generally similar to those of the survey item determination unit 110, the survey answer management unit 120, the communication unit 130, and the control unit 140 of the urination survey system 100. Here, at least a part of the application may be replaced with a hardware device or a firmware device that may perform a substantially equal or equivalent function, as necessary.

Further, the communication network 300 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 300 described herein may be the Internet or the World Wide Web (WWW). However, the communication network 300 is not necessarily limited thereto, and may at least partially include known wired/wireless data communication networks, known telephone networks, or known wired/wireless television communication networks.

For example, the communication network 300 may be a wireless data communication network, at least a part of which may be implemented with a conventional communication scheme such as WiFi communication, WiFi-Direct communication, Long Term Evolution (LTE) communication, Bluetooth communication (more specifically, Bluetooth Low Energy (BLE) communication), infrared communication, and ultrasonic communication.

First, according to one embodiment of the invention, the urination survey system 100 may detect whether an event related to the user's urination occurs, on the basis of a sound signal related to the user's urination acquired from the device 200 of the user.

Further, when the event related to the user's urination occurs, the urination survey system 100 according to one embodiment of the invention may determine at least one survey item to be provided to the user, with reference to context information on the event.

For example, referring to FIG. 3, the urination survey system 100 according to one embodiment of the invention may determine the IPSS survey items as a plurality of survey items related to the user's urinary health, and may determine context information indicating that the user's urinary stream is weak, when there occurs an event in which a maximum urine flow rate estimated from the sound signal related to the user's urination is 15 ml/sec or less. Further, the urination survey system 100 according to one embodiment of the invention may determine at least one survey item 310 to be provided to the user among the IPSS survey items, with reference to the context information.

Next, the urination survey system 100 according to one embodiment of the invention may provide the at least one survey item 310 to the device 200 of the user.

Next, the urination survey system 100 according to one embodiment of the invention may acquire the user's answer corresponding to the at least one survey item 310 from the device 200 of the user.

Meanwhile, the urination survey system 100 according to one embodiment of the invention may estimate an answer corresponding to at least one survey item 320 other than the at least one determined survey item 310, among the plurality of survey items (i.e., the IPSS survey items), with reference to the context information on the event related to the urination.

For example, when the user's urination is started at 11 p.m. and the user's bedtime acquired from the device 200 of the user is 10 p.m., the urination survey system 100 according to one embodiment of the invention may estimate an answer corresponding to the at least one survey item 320 other than the at least one determined survey item 310, among the IPSS survey items, with reference to the context information indicating that the urination is nocturia. That is, the urination survey system 100 according to one embodiment of the invention may estimate an answer corresponding to the at least one survey item 320 by calculating the number of occurrences of the user's nocturia during a predetermined period of time (e.g., a week).

Next, the urination survey system 100 according to one embodiment of the invention may calculate answer scores for the IPSS survey items at a predetermined period or at each time of urination, with reference to the user's answer corresponding to the at least one survey item 310 and the estimated answer, and may provide the device 200 of the user with an answer analysis result such as an average, a maximum value, or a minimum value of the scores.

Meanwhile, according to another embodiment of the invention, when the occurrence of the event related to the user's urination is detected on the basis of various signals such as a sound or video signal related to the user's urination, the signals may be analyzed to estimate information on at least one of a urine flow rate (or a voided urine volume per hour), a start point of the urination, an end point of the urination, and a voided urine volume of the user, and a voiding diary of the user may be generated on the basis of the estimated information. According to another embodiment of the invention, the voiding diary may include information on not only a urine flow rate, a urination duration (i.e., a difference between start and end points of the urination), and a voided urine volume by day or hour, but also an average urine flow rate, an average urination duration, an average voided urine volume, a maximum urine flow rate, a maximum urination duration, and a maximum voided urine volume calculated by statistically analyzing at least one of the urine flow rate, urination duration, and voided urine volume. For example, according to another embodiment of the invention, the information on at least one of the urine flow rate (or voided urine volume per hour), start point of the urination, end point of the urination, and voided urine volume of the user may be calculated on the basis of urination parameters related to an effective (or RMS) value and a spectral centroid calculated from the sound signal related to the user's urination.

Further, according to another embodiment of the invention, an analysis comment on the user's urination may be generated with reference to the generated voiding diary.

For example, according to another embodiment of the invention, a urination pattern appearing in the voiding diary of the user and a predetermined reference urination pattern may be compared and analyzed to specify abnormal urination of the user, and information on a time when the abnormal urination occurs, a urine flow rate of the abnormal urination, a voided urine volume of the abnormal urination, and a reason for specifying the abnormal urination may be generated as an analysis comment on the urination. Meanwhile, according to another embodiment of the invention, the analysis comment on the urination may be partially similar to a conventional chart used for a medical insurance claim.

The embodiments according to the invention as described above may be implemented in the form of program instructions that can be executed by various computer components, and may be stored on a computer-readable recording medium. The computer-readable recording medium may include program instructions, data files, and data structures, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler, but also high-level language codes that can be executed by a computer using an interpreter. The above hardware devices may be changed to one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for conducting a survey on urination, the method comprising the steps of:
determining at least one survey item to be provided to a user when an event related to the user's urination occurs, with reference to context information on the event; and
acquiring the user's answer corresponding to the at least one survey item.

2. The method of claim 1, wherein in the determining step, the event related to the urination is recognized on the basis of a sound signal related to the user's urination.

3. The method of claim 1, wherein the determining step comprises the step of estimating an answer corresponding to at least one survey item other than the at least one determined survey item, among a plurality of survey items related to the user's urinary health, with reference to the context information on the event.

4. The method of claim 1, wherein the acquiring step comprises the step of providing the user with the at least one survey item when the event is ended.

5. The method of claim 1, wherein the acquiring step comprises the step of providing the user with the at least one survey item and an estimated answer corresponding to the at least one survey item.

6. The method of claim 1, wherein in the acquiring step, the user's urinary health status is determined with reference to the user's answer corresponding to the at least one survey item.

7. The method of claim 1, wherein the context information on the event includes information on at least one of a start or end point of the event, circumstances of the event, and a pattern of occurrence of the event.

8. A non-transitory computer-readable recording medium having stored thereon a computer program for executing the method of claim 1.

9. A system for conducting a survey on urination, the system comprising:
a survey item determination unit configured to determine at least one survey item to be provided to a user when an event related to the user's urination occurs, with reference to context information on the event; and
an answer acquisition unit configured to acquire the user's answer corresponding to the at least one survey item.
